(19) United States
(12) Patent Application Publication
(10) Pub. No.: US 2021/0059864 A1
(43) Pub. Date: Mar. 4, 2021

(54) ENGINEERED KNITTED GARMENT HAVING INTEGRATED COMPRESSION ZONES

(71) Applicant: LULULEMON ATHLETICA CANADA INC., Vancouver (CA)

(72) Inventors: Audrey Milligan REILLY, Vancouver (CA); Sophie Adrienne DOYLE, Vancouver (CA); Alison Claire MAXFIELD, Halfmoon Bay (CA); Joshua David HINEY, San Francisco, CA (US); Yu Wen Angela HUANG, Vancouver (CA); Sisi JIANG, Vancouver (CA); Eliot Jean Cohen PIRENNE, Vancouver (CA)

(21) Appl. No.: 17/006,177

(22) Filed: Aug. 28, 2020

Related U.S. Application Data
(60) Provisional application No. 62/894,292, filed on Aug. 30, 2019.

(51) Int. Cl. A61F 13/08; A41D 1/08

(52) U.S. Cl. CPC A61F 13/08; A41D 2500/10; A41D 1/08

(57) ABSTRACT

Examples of an engineered knitted garment with a number of integrated compression zones are disclosed. The engineered knitted garment may be a pair of tights having a first compression zone located at the calves, a second compression zone located at the knees, and a third compression zone located at the waist. The compressive moduli of the compression zones may be different to provide higher compression in areas where greater support is required (e.g., at the calves) and lower compression in areas where greater mobility is required (e.g., at the knees).

This application claims priority from and the benefit of the filing date of U.S. Provisional Patent Application No. 62/894,292, filed Aug. 30, 2019, and the entire content of such application is incorporated herein by reference.

### TECHNICAL FIELD

This application relates to the field of garments, and more specifically, to an engineered knitted garment having integrated compression zones for sporting activities, exercise, and the like.

### BACKGROUND

Athletes and consumers are demanding high performance activewear and sportswear, such as for example, compression sportswear. Studies have found that compression garments can provide benefits such as reducing muscle fatigue, providing muscle support, reducing muscle vibration, reducing the risk of strain, preventing injuries, etc. Currently, most compression garments are made by cutting and sewing fabrics with different structural properties to provide different levels of compression. However, such garments typically have a number of seams located throughout the garment that may cause chaffing. In addition, making such garments typically involves greater fabric waste and increased labor and manufacturing costs.

### SUMMARY OF THE APPLICATION

In one aspect, an engineered knitted garment such as a pair of tights is provided having at least a first compression zone located at the calves, a second compression zone located at the knees, and a third compression zone located at the waist. The compressive moduli of the compression zones may be different to provide higher compression in areas where greater support is required (e.g., at the calves) and lower compression in areas where greater mobility is required (e.g., at the knees).

In addition to the aspects and embodiments described above, further aspects and embodiments will become apparent by reference to the drawings and study of the following detailed description.

### BRIEF DESCRIPTION OF THE DRAWINGS

Throughout the drawings, reference numbers may be re-used to indicate correspondence between referenced elements. The drawings are provided to illustrate example embodiments described herein and are not intended to limit the scope of the application. Sizes and relative positions of elements in the drawings are not necessarily drawn to scale. For example, the shapes of various elements and angles may be not drawn to scale, and some of these elements may be arbitrarily enlarged and positioned to improve drawing legibility.

FIG. 1 is a front view illustrating an example engineered knitted garment having integrated compression zones.

FIG. 2 is a rear view thereof.

FIG. 3 is a front view thereof illustrating example compression zones.

FIG. 4 is a rear view thereof.

FIG. 5 is a left side view illustrating an example knitted panel for the engineered knitted garment of FIGS. 1-4.

FIG. 6 is a front view illustrating another example engineered knitted garment having integrated mesh texture compression zones.

FIG. 7 is a rear view thereof.

FIG. 8 includes left, right, front, and rear views illustrating example knitted panels for the engineered knitted garment of FIGS. 6-7.

FIG. 9 is a block diagram illustrating an example warp knitting machine.

FIG. 10 is a front view illustrating another example engineered knitted garment having integrated compression zones.

FIG. 11 is a rear view thereof.

FIG. 12 is a front view illustrating another example engineered knitted garment having integrated compression zones.

FIG. 13 is a rear view thereof.

FIG. 14 is a front view illustrating another example engineered knitted garment having integrated compression zones.

FIG. 15 is a front view illustrating another example engineered knitted garment having integrated compression zones.

FIG. 16 is a front view illustrating another example engineered knitted garment having integrated compression zones.

FIG. 17 is a rear view thereof.

FIGS. 18-22 are front, side, and rear views illustrating additional example engineered knitted garments having integrated compression zones.

### DETAILED DESCRIPTION

In the following description, details are set forth to provide an understanding of the application. In some instances, certain structures, techniques, and methods have not been described or shown in detail in order not to obscure the application.

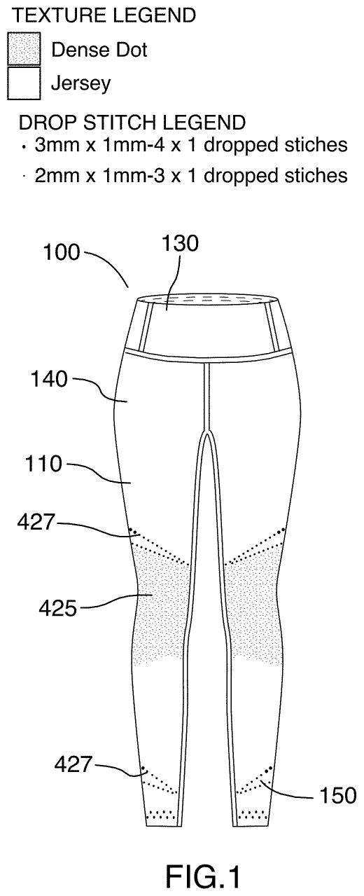

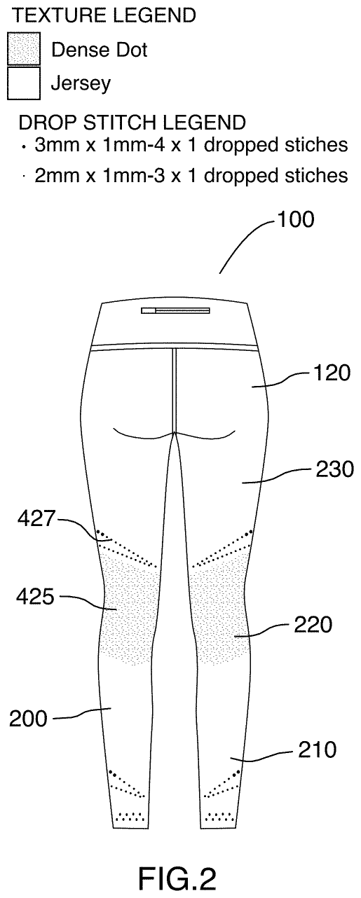

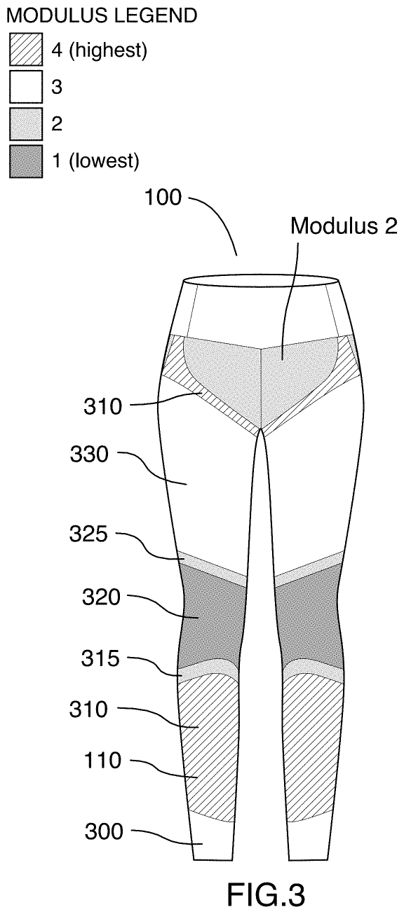

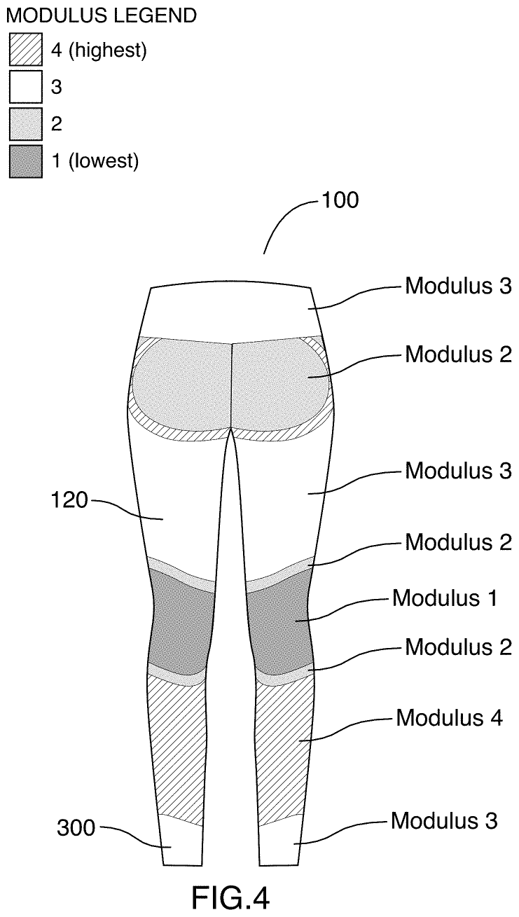

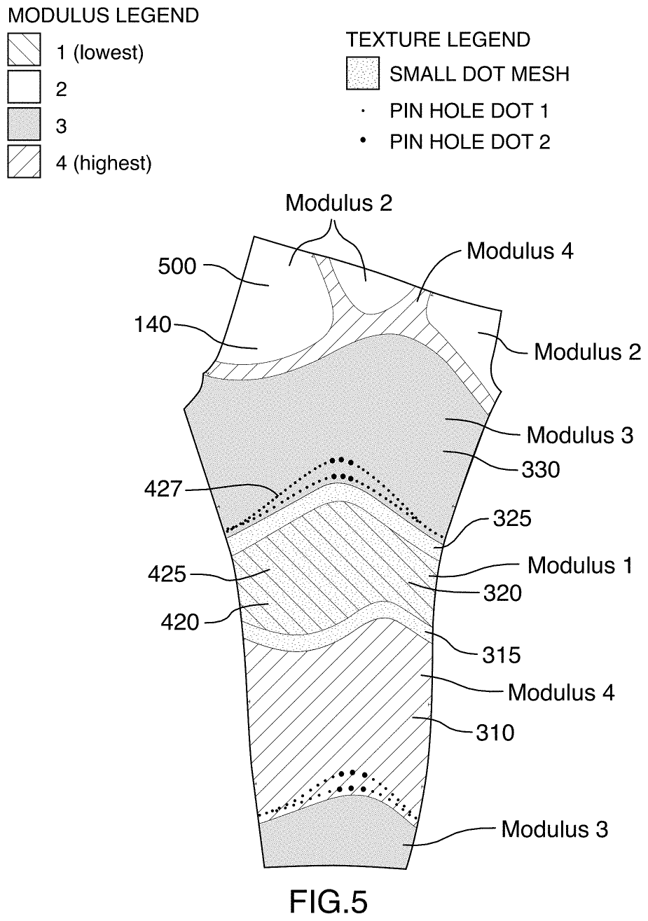

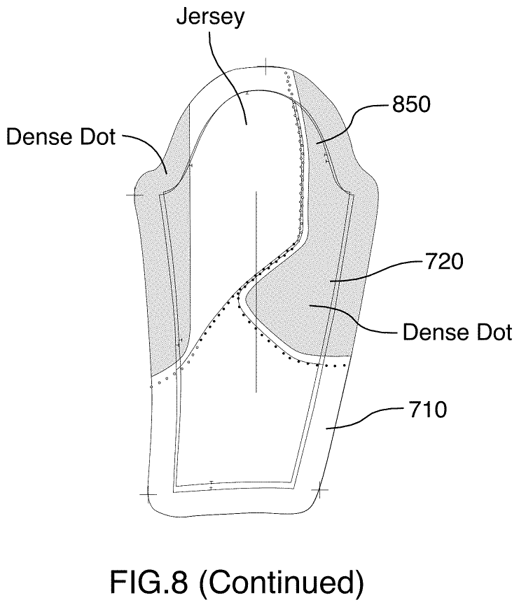

FIG. 1 is a front view illustrating an example engineered knitted garment 100 having integrated compression zones 300. FIG. 2 is a rear view thereof. FIG. 3 is a front view thereof illustrating example compressive zones 300. FIG. 4 is a rear view thereof. FIG. 5 is a left side view of an example knitted panel 500 for the engineered knitted garment 100 of FIGS. 1-4. And, FIG. 9 is a block diagram illustrating an example warp knitting machine 900.

According to one embodiment of the application, there is provided an engineered knitted garment 100 having integrated compression zones 300. The garment 100 may be a compression tight or pair of tights. Of course, the garment 100 may be any other garment such as a bra, top, shirt, jacket, skirt, skort, pants, shorts, bathing suits, etc. The garment 100 has a front side 110 and a rear side 120, a waistband 130, and first and second or left and right legs 140, 150. Each leg 140, 150 of the garment 100 may be formed from a knitted panel 500 (such as the left knitted panel 500 shown in FIG. 5).

The knitted panel 500 is shaped and sized to cover a portion of a wearer's body (e.g., the wearer's leg, a portion of the wearer's torso, etc.) and may be knitted using a raschel knitting machine 900 having two jacquard guide bars 910, 920. An example of such a machine is the Rascheltronic™ 4/2 jacquard raschel warp knitting machine available form Karl Mayer Holding GmbH & Co. KG. The two jacquard guide bars 910, 920 are used for scamlessly knitting different compression zones 300. One of the guide bars (e.g., 910) may be used to provide a desired combination of stiches and knit constructions 200 while the other guide bar (e.g., 920) may be used to adds extra yarn (e.g., jacquards in elastane) to change the density and/or weight in a particular zone such that the weight difference between the lowest compression zone and the highest compression zone may be around 200 grams per square meter (GSM). Typically, this cannot be obtained with a single jacquard bar knitting machine. Different levels (e.g., 3 or 4) of elastane content (or similar synthetic fibers or yarn such as Spandex™, Lycra™, polyamide, polyether-polyurea copolymer, etc.) may be engineered into the fabric of the knitted panel 500 to create different compression zones (e.g., 3 or 4) 300. Mesh textures 425 for ventilation zones 420 may also be included. The mesh textures 425 may be used to provide lower compression zones 300 in selected areas. Further compression zones 300 may be created by combining elastane jacquard with jacquard patterns which provides a double effect with respect to compression.

According to one embodiment, the knitted panel 500 may include a first compression zone 310 having a first knit stitch construction (or pattern) (e.g., Jersey knit, etc.) 210. One of the jacquard guide bars 910 of the knitting machine 900 may be used to define the first knit stitch construction 210 and the other of the jacquard guide bars 920 may be used to add an elastane jacquard construction (or pattern) to the first knit stitch construction 210 to increase fabric weight in the first compression zone 310. The first compression zone 310 may have a first amount of compression (or compressive modulus).

The knitted panel 500 may include a second compression zone 320 joined to the first compression zone 310 by a seamless transition 315 and may have a second knit stitch construction (or pattern) (e.g., Dense Dot knit, etc.) 220. The second compression zone 320 may have a second amount of compression (or compressive modulus). According to one embodiment, the first amount of compression of the first compression zone 310 may be greater than the second amount of compression of the second compression zone 320. That is, the first compression zone 310 may provide a tighter or more compressive fit that the second compression zone 320. Advantageously, the seamless joining of the first and second compression zones 310, 320 reduces chaffing.

According to one embodiment, a difference in fabric weight between the first compression zone 310 and the second compression zone 320 may be between 100 and 220 grams per square meter (GSM). According to one embodiment, the first and second knit stitch constructions 210, 220 of the garment 100 may be the same. According to one embodiment, at least one of the first and second knit stitch constructions 210, 220 may be a jacquard pattern.

For example, the second knit stitch construction 220 may include an elastane jacquard construction (or pattern) that is added to the second knit stitch construction 220 using the other of the jacquard guide bars. According to one embodiment, an amount (or extent) of the elastane jacquard in the first knit stitch construction 210 may be greater than an amount (or extent) of the elastane jacquard in the second knit stitch construction 220.

According to one embodiment, the engineered knitted garment 100 may further include a third compression zone 330 joined to at least one of the first compression zone 310 and the second compression zone 320 by a respective seamless transition 325 and having a third knit stitch construction (or pattern) (e.g. Jersey knit, etc.) 230. One of the jacquard guide bars of the knitting machine may be used to define the third knit stitch construction 230. The third compression zone 330 may have a third amount of compression (or compressive modulus). According to one embodiment, the third amount of compression of the third compression zone 330 may be greater than the second amount of compression of the second compression zone 320 but less than the first amount of compression of the first compression zone 310. The third knit stitch construction 230 may be a jacquard pattern. The third compression zone 330 may include an elastane jacquard construction (or pattern) added to the third knit stitch construction 230 using the other of the jacquard guide bars. According to one embodiment, an amount (or extent) of the elastane jacquard in the third compression zone 330 may be less than an amount of elastane jacquard in the first compression zone 310.

According to one embodiment, at least a portion of the second compression zone 320 may include a mesh texture or pattern 425. According to another embodiment, the garment 100 may further include at least one ventilation zone 420 seamlessly knitted into the knitted panel 500. The ventilation zone 420 may be formed from or include a mesh 425 or a pattern of drop stitch open holes 427. Advantageously, sheer and open hole mesh textures 425, 427 may be knit into the knitted panel 500 to improve breathability, to achieve a lower compressive modulus, to reduce restriction, and/or for visual effect.

As mentioned above, according to one embodiment as shown in FIGS. 1-4, the engineered knitted garment 100 may be a compression tight (or pair of compression tights or tights). In this embodiment, the first compression zone 310 may be located in one or more of a calf area, an under buttock area, and an upper waistband area with anchoring portions to respective hip areas of the compression tight 100. The second compression zone 320 may be located in a knee area of the compression tight 100. The garment 100 may further include a third compression zone 330 joined to at least one of the first compression zone 310 and the second compression zone 320 by a respective seamless transition 325 and having a third knit stitch construction 230 wherein one of the jacquard guide bars is used to define the third knit stitch construction 230, the third compression zone 330 having a third amount of compression (or compressive modulus), the third amount of compression being greater than the second amount of compression of the second compression zone 320 but less than the first amount of compression of the first compression zone 310. The third compression zone 330 may be located in one or more of a pelvic area and an area at least partially surrounding a knee area of the compression tight 100. Advantageously, the compressive moduli of the compression zones 300 may be different to provide higher compression in areas where greater support is required (e.g., at the calves) and lower compression in areas where greater mobility is required (e.g., at the knees). The high modulus in the under buttock and upper waistband areas anchors the compression tight 100 to the waistband and reduces crotch sagging and bum jiggle. The low modulus in the knee area of the compression tight 100 reduces restriction and bulk. And, the gradate levels of compression in the transition 325 provide a distraction free fit.

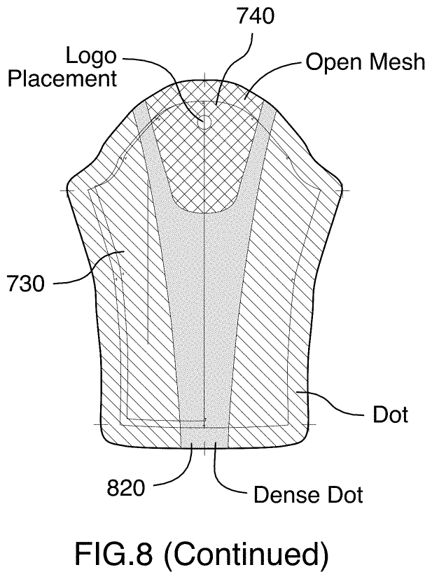

In some embodiments, the garment 100 may have more than three compression zones 300. For example, FIGS. 3 and 4 illustrate a garment 100 (e.g., a pair of tights) having 4 compression zones. As described further below, FIGS. 10 to 13 also illustrate garments 1000, 1200 having 4 compression zones.

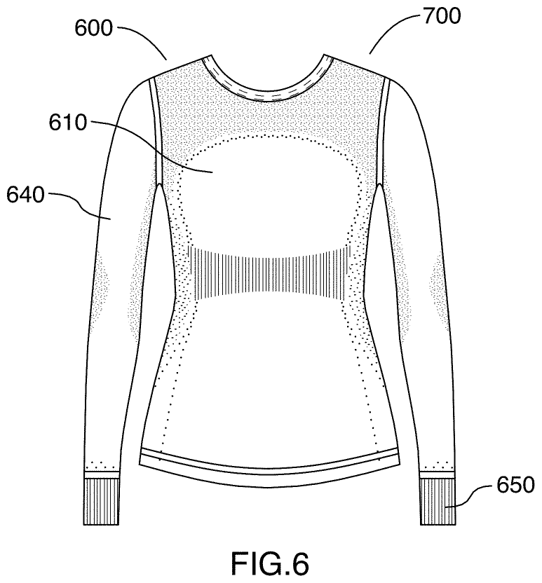

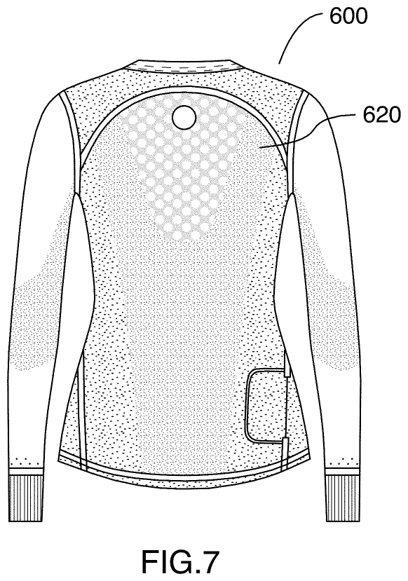

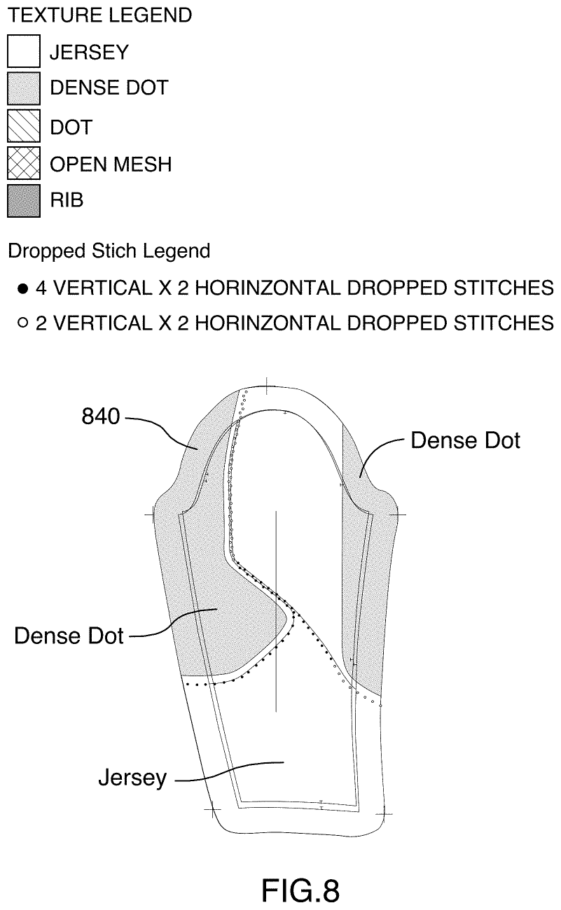

FIG. 6 is a front view illustrating another example engineered knitted garment 600 having integrated mesh texture compression zones 700. FIG. 7 is a rear view thereof. And, FIG. 8 includes left, right, front, and rear views illustrating example knitted panels 800 for the engineered knitted garment 600 of FIGS. 6-7.

As mentioned above, the engineered knitted garment of the present application may be any garment such as a bra, top, shirt, jacket, skirt, skort, pants, shorts, bathing suits, etc., in addition to the tights 100 shown in FIGS. 1-4. For example, FIG. 6 shows an engineered knitted garment 600 which is a top or shirt. The garment 600 has a front side 610, a rear side 620, and first and second or left and right arms 640, 650. Each arm 640, 650 and the front and rear sides 610, 620 of the garment 600 may be formed from a respective knitted panel 800 (such as the knitted panels 810, 820, 840, 850 shown in FIG. 8).

The garment 600 has multiple compression zones 700 that are formed from different mesh textures. The different mesh textures provide different levels of breathability (or ventilation) and different levels (or moduli) of compression. For example, the wrist area of each arm 640, 650 and the lower area on the front side 610 of the garment 600 may have a jersey texture providing a first compression zone 710. The elbow area of each arm 640, 650, the under neck area on the front side 610, and the middle back area on the rear side 620 of the garment 600 may have a dense dot texture providing a second compression zone 720. The underarm areas on the sides of the rear side 620 of the garment 600 may have a dot texture providing a third compression zone 730. The upper back area on the rear side 620 of the garment 600 may have an open mesh texture providing a fourth compression zone 740. And, the middle area on the front side 610 of the garment 600 may have a rib texture providing a fifth compression zone 750. For example, the first compression zone 710 (at the wrists) may have a higher compressive modulus than the second compression zone 720 (at the elbows) to improve fit and user comfort.

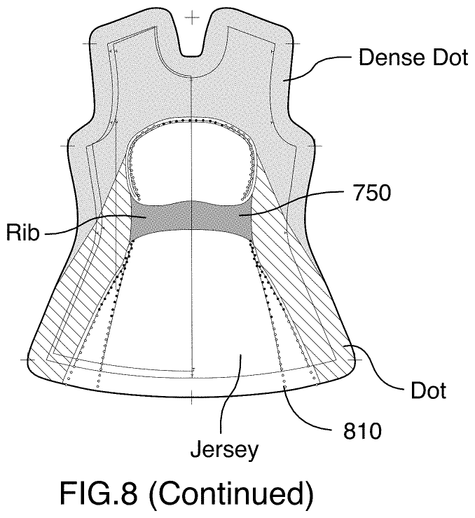

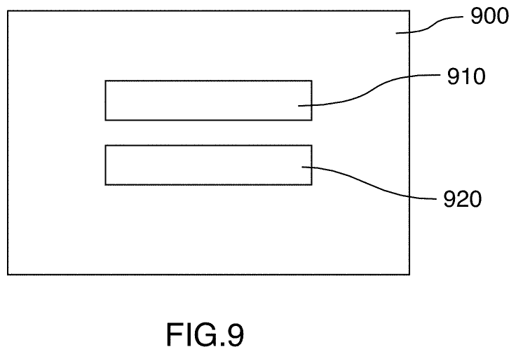

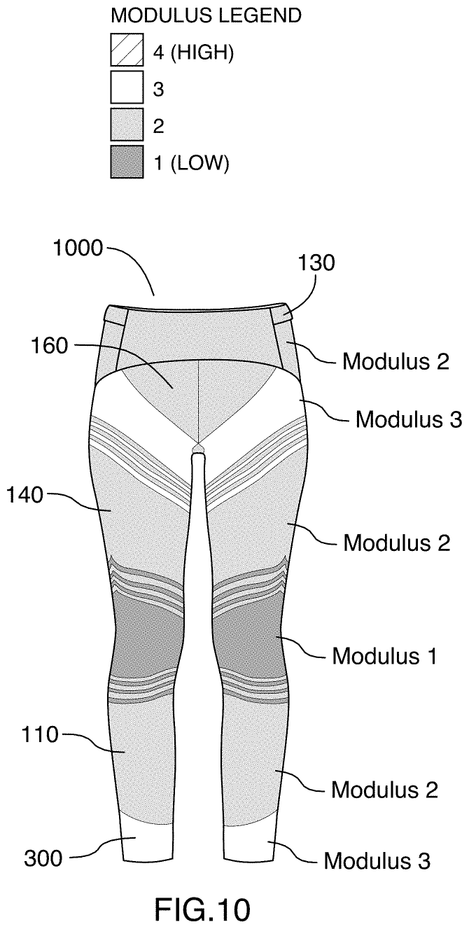

FIG. 10 is a front view illustrating another example engineered knitted garment 1000 having integrated compression zones 300. FIG. 11 is a rear view thereof. FIG. 12 is a front view illustrating another example engineered knitted garment 1200 having integrated compression zones 300. And, FIG. 13 is a rear view thereof. Similar to the engineered knitted garment 100 of FIGS. 3-4, the engineered knitted garments 1100, 1200 of FIGS. 10-11 and FIGS. 12-13 may be compression tights or pairs of tights for women and men, respectively. The garments 1100, 1200 each have a front side 110, a rear side 120, a waistband 130, and first and second or left and right legs 140, 150. The garments 1100, 1200 include a number (e.g., 4) of compression zones 300 each having a respective amount of compression (or compressive modulus)(e.g., Modulus 1, Modulus 2, etc.). The layout or arrangement of the compression zones 300 in the pelvic area 160 of the garments 1100, 1200 is tuned to match the sex of the wearer to provide support for sensitive areas. For example, and referring to FIG. 10 which shows the women's garment 1000, less support (i.e., a lower compressive modulus Modulus 2) may be provided in the genital area and greater support (i.e., a higher compressive modulus Modulus 3) may be provided around the genital area. As another example, and referring to FIG. 12 which shows the men's garment 1200, greater support (i.e., a higher compressive modulus Modulus 4) may be provided in the genital area and less support (i.e., a lower compressive modulus Modulus 2) may be provided around the genital area. The seamless transitions 1015, 1025 between compression zones 300 may be formed by alternating bands (e.g., 3 bands) of fabric from each of the adjoining compression zones 300. The garment 1100, 1200 may be of light weight being made from, for example, lighter 60 dtex elastane rather than, for example, the heavier 78 dtex elastane that may be used in the garment 100 of FIGS. 3-4. This construction is advantageous for use in running activities.

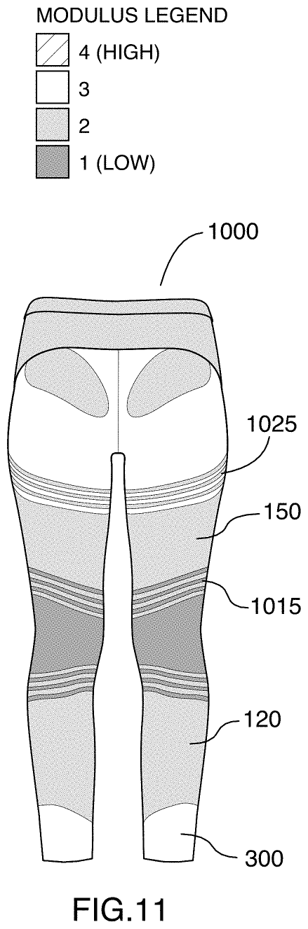

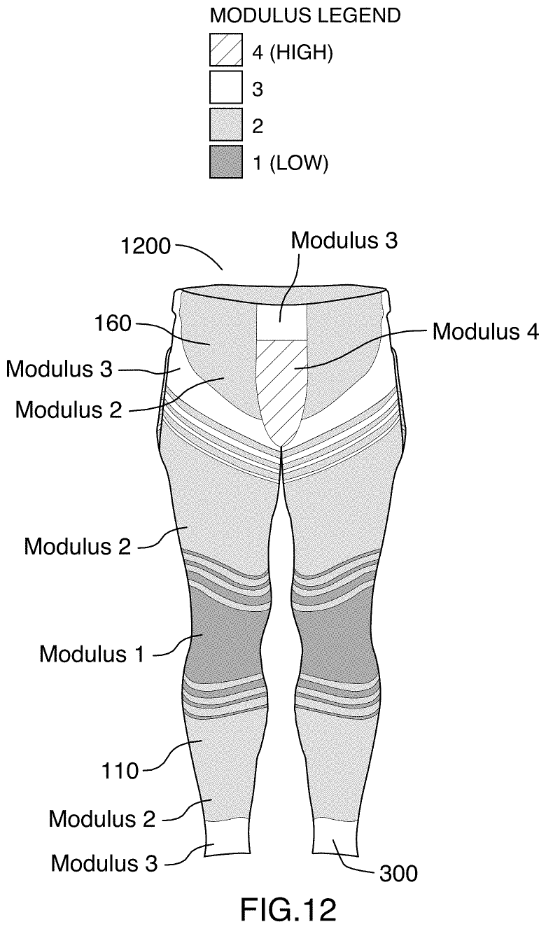

FIG. 14 is a front view illustrating another example engineered knitted garment 1400 having integrated compression zones 300. And, FIG. 15 is a front view illustrating another example engineered knitted garment 1500 having integrated compression zones 300. The engineered knitted garment 1400 of FIG. 14 may be a sports bra or top and the engineered knitted garment 1500 of FIG. 15 may be compression tights or a pair of tights. The garments 1400, 1500 include a number (e.g., 3) of compression zones 300 each having a respective amount of compression (or compressive modulus) (e.g., Modulus 1, Modulus 2, etc.). The layout or arrangement of the compression zones 300 in the breast area 170 of the garment 1400 of FIG. 14 is tuned to provide support for sensitive areas. For example, greater support (i.e., a higher compressive modulus Modulus 3) may be provided in the areola/nipple area, and on either side of the breast tissue, and less support (i.e., a lower compressive modulus Modulus 1) may be provided around the areola/nipple area.

The layout of the compression zones 300 in the joint areas 180 (e.g., in the knee area) of the garment 1500 of FIG. 15 is tuned to provide greater support (i.e., a higher compressive modulus Modulus 3) around the joint and less support (i.e., a lower compressive modulus Modulus 1) at the joint. This provides the wearer with an increased feeling of support and awareness in the joint areas 180. Such garments may be used in training activities where additional support around the joints is desired. The garments 1400, 1500 may be of light weight and may be made using cooling yarn which is advantageous for use in training activities.

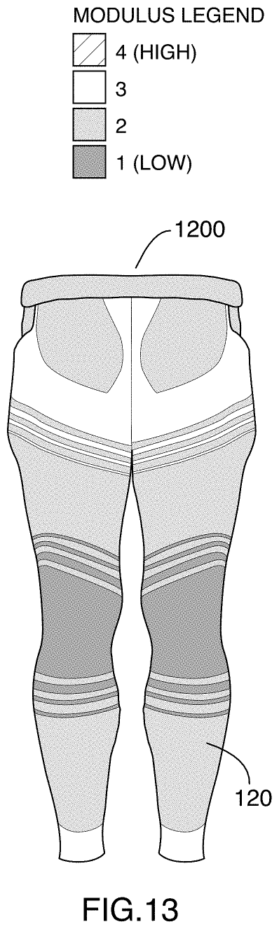

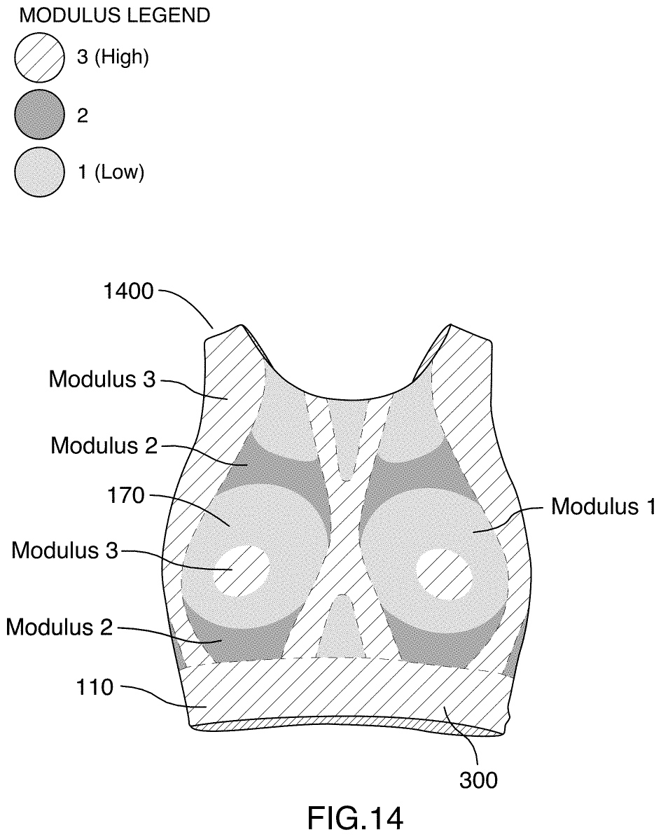

FIG. 16 is a front view illustrating another example engineered knitted garment 1600 having integrated compression zones 300. And, FIG. 17 is a rear view thereof. The engineered knitted garment 1600 of FIGS. 16-17 be a bathing suit or leotard. The garment 1600 includes a number (e.g., 3 or 4) of compression zones 300 each having a respective amount of compression (or compressive modulus) (e.g., Modulus 1, Modulus 2, etc.). The layout or arrangement of the compression zones 300 in the abdominal area 190 of the garment 1600 of FIG. 16 is tuned to provide support where required. For example, to improve flexibility, less support (i.e., a lower compressive modulus Modulus 2) may be provided in the central area of the abdomen and greater support (i.e., a higher compressive modulus Modulus 3) may be provided around the central area.

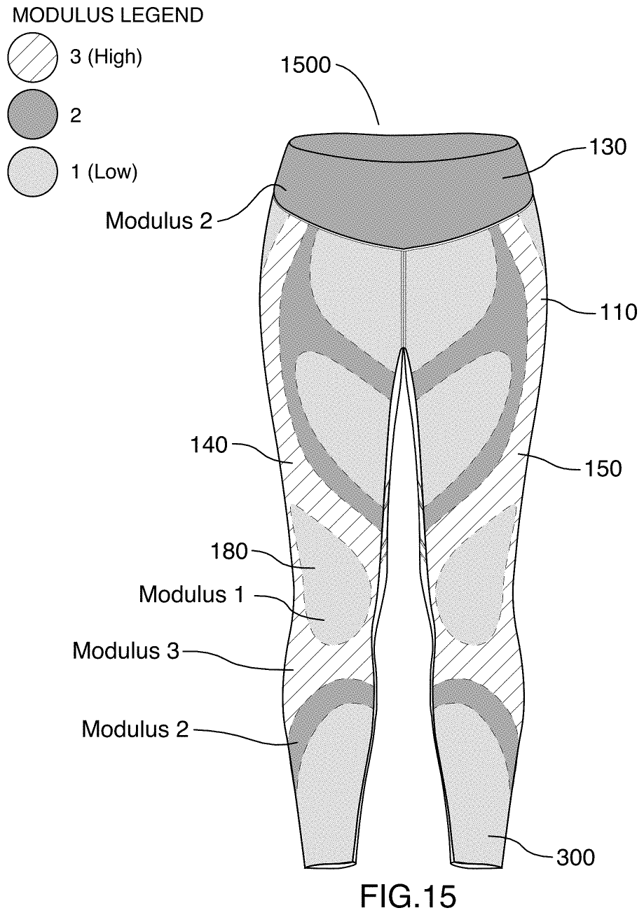

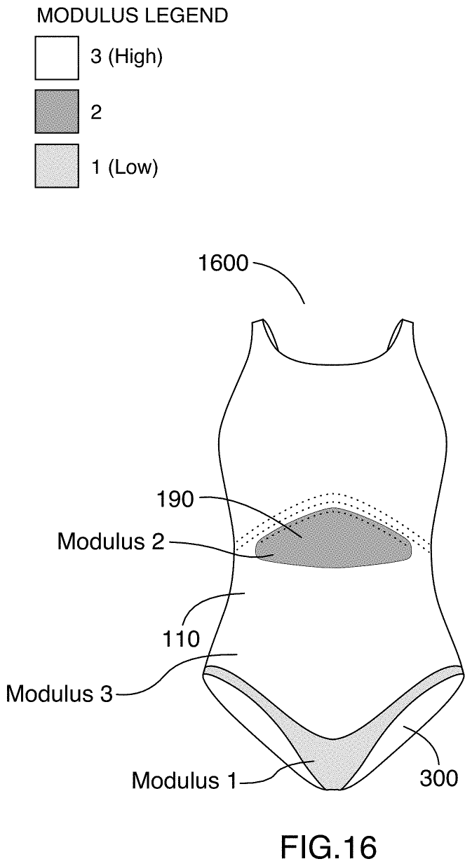

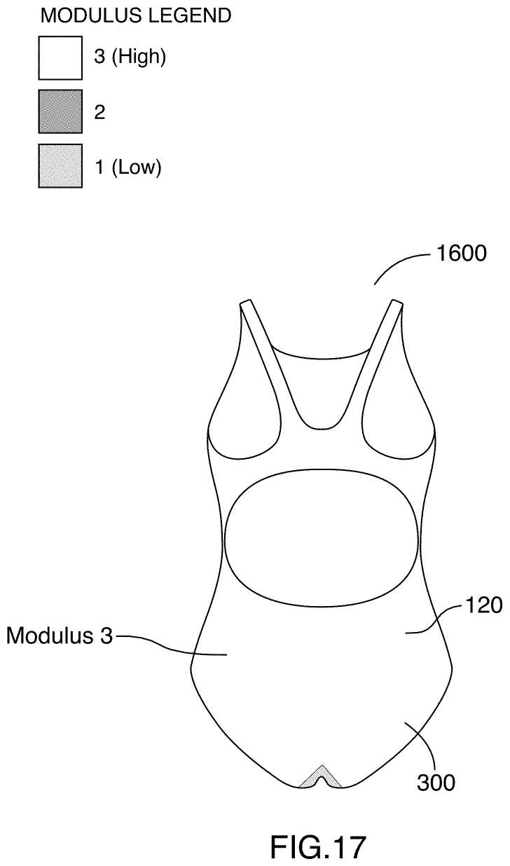

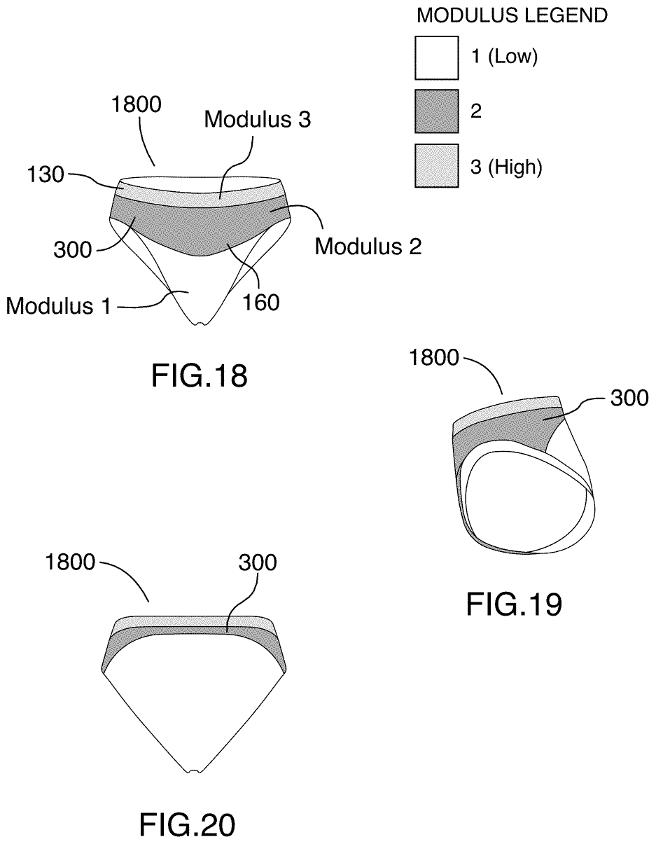

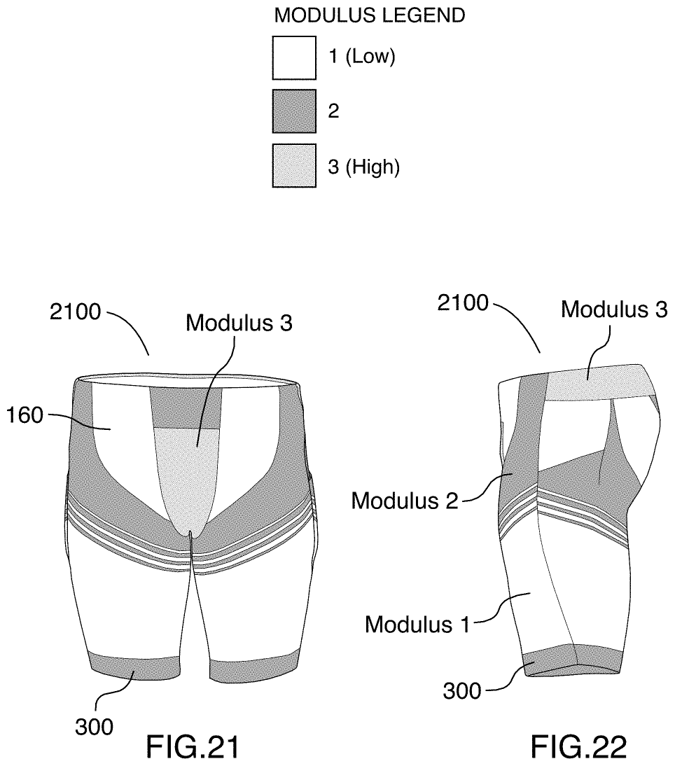

FIGS. 18-22 are front, side, and rear views illustrating additional example engineered knitted garments 1800, 2100 having integrated compression zones 300. The engineered knitted garments 1800, 2100 of FIGS. 18-20 and FIGS. 21-22 may be underwear for women and men, respectively. The garments 1800, 2100 include a number (e.g., 3 or 4) of compression zones 300 each having a respective amount of compression (or compressive modulus) (e.g., Modulus 1, Modulus 2, etc.). Similar to the garments 1100, 1200 of FIGS. 10-13, the layout or arrangement of the compression zones 300 in the pelvic area 160 of the garments 1800, 2100 is tuned to match the sex of the wearer to provide support where required for sensitive areas. In addition, and referring to FIGS. 18-20 which show the women's garment 1800, greater support (i.e., a higher compressive modulus Modulus 3) may be provided in the waistband area or waistband 130 to replicate or approximate the hugged feeling provided by a pair of compression tights and less support (i.e., a lower compressive modulus Modulus 2) may be provide in the pelvic area 160 below the waistband 130.

In some embodiments, the compression zones 300 may be made from a blend of natural and synthetic fabrics.

While particular elements, embodiments and applications of the present application have been shown and described, it will be understood, that the scope of the application is not limited thereto, since modifications can be made by those skilled in the art without departing from the scope of the present application, particularly in light of the foregoing teachings. Thus, for example, in any method or process disclosed herein, the acts or operations making up the method/process may be performed in any suitable sequence and are not necessarily limited to any particular disclosed sequence. Elements and components can be configured or arranged differently, combined, and/or eliminated in various embodiments. The various features and processes described above may be used independently of one another, or may be combined in various ways. All possible combinations and subcombinations are intended to fall within the scope of this application. Reference throughout this disclosure to “some embodiments,” “an embodiment,” or the like, means that a particular feature, structure, step, process, or characteristic described in connection with the embodiment is included in at least one embodiment. Thus, appearances of the phrases “in some embodiments,” “in an embodiment,” or the like, throughout this disclosure are not necessarily all referring to the same embodiment and may refer to one or more of the same or different embodiments. Indeed, the novel methods and systems described herein may be embodied in a variety of other forms; furthermore, various omissions, additions, substitutions, equivalents, rearrangements, and changes in the form of the embodiments described herein may be made without departing from the spirit of the application.

Various aspects and advantages of the embodiments have been described where appropriate. It is to be understood that not necessarily all such aspects or advantages may be achieved in accordance with any particular embodiment. Thus, for example, it should be recognized that the various embodiments may be carried out in a manner that achieves or optimizes one advantage or group of advantages as taught herein without necessarily achieving other aspects or advantages as may be taught or suggested herein.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without operator input or prompting, whether these features, elements and/or steps are included or are to be performed in any particular embodiment. No single feature or group of features is required for or indispensable to any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

The example calculations, simulations, results, graphs, values, and parameters of the embodiments described herein are intended to illustrate and not to limit the disclosed embodiments. Other embodiments can be configured and/or operated differently than the illustrative examples described herein.

What is claimed is:

1. An engineered knitted garment having integrated compression zones, comprising:
a knitted panel shaped and sized to cover a portion of a wearer's body, the knitted panel being knitted using a raschel knitting machine having two jacquard guide bars, the knitted panel comprising:
a first compression zone having a first knit stitch construction wherein one of the jacquard guide bars is used to define the first knit stitch construction and the other of the jacquard guide bars is used to add an elastane jacquard to the first knit stitch construction to increase fabric weight in the first compression zone, the first compression zone having a first amount of compression; and,
a second compression zone joined to the first compression zone by a seamless transition and having a second knit stitch construction, the second compression zone having a second amount of compression;
wherein the first amount of compression is greater than the second amount of compression.

2. The engineered knitted garment of claim 1, wherein a difference in fabric weight between the first compression zone and the second compression zone is between 100 and 220 grams per square meter (GSM).

3. The engineered knitted garment of claim 1, wherein the first and second knit stitch constructions are the same.

4. The engineered knitted garment of claim 1, wherein at least one of the first and second knit stitch constructions is a jacquard pattern.

5. The engineered knitted garment of claim 1, wherein the second knit stitch construction includes an elastane jacquard that is added to the second knit stitch construction by the other of the two jacquard guide bars, an amount of the elastane jacquard added in the first knit stitch construction being greater than an amount of the elastane jacquard added in the second knit stitch construction.

6. The engineered knitted garment of claim 1, further comprising a third compression zone joined to at least one of the first compression zone and the second compression zone by a respective seamless transition and having a third knit stitch construction wherein one of the jacquard guide bars is used to define the third knit stitch construction, the third compression zone having a third amount of compression, the third amount of compression being greater than the second amount of compression of the second compression zone but less than the first amount of compression of the first compression zone.

7. The engineered knitted garment of claim 6, wherein the third knit stitch construction is a jacquard pattern.

8. The engineered knitted garment of claim 6, wherein the third compression zone further includes an elastane jacquard added to the third knit stitch construction using the other of the jacquard guide bar, an amount of the elastane jacquard in the third compression zone being less than an amount of elastane jacquard in the first compression zone.

9. The engineered knitted garment of claim 1, wherein at least a portion of the second compression zone has a mesh texture.

10. The engineered knitted garment of claim 1, further comprising at least one ventilation zone seamlessly knitted into the knitted panel.

11. The engineered knitted garment of claim 1, wherein the garment is a compression tight or compression tights.

12. The engineered knitted garment of claim 11, wherein the first compression zone is located in one or more of a calf area, an under buttock area, and an upper waistband area with anchoring portions to respective hip areas of the compression tight.

13. The engineered knitted garment of claim 11, wherein the second compression zone is located in a knee area of the compression tight.

14. The engineered knitted garment of claim 11, further comprising a third compression zone joined to at least one of the first compression zone and the second compression zone by a respective seamless transition and having a third knit stitch construction wherein one of the jacquard guide bars is used to define the third knit stitch construction, the third compression zone having a third amount of compression, the third amount of compression being greater than the second amount of compression of the second compression zone but less than the first amount of compression of the first compression zone.

15. The engineered knitted garment of claim 14, wherein the third compression zone is located in one or more of a pelvic area and an area at least partially surrounding a knee area of the compression tight.

16. The engineer knitted garment of claim 1, wherein the raschel knitting machine is a warp knitting machine.

17. The engineered knitted garment of claim 1, wherein the seamless transition is formed from one or more alternating bands of fabric from the first and second compression zones.

18. The engineered knitted garment of claim 1, wherein the seamless transition has an amount of compression between that of the first and second amounts of compression.

19. The engineered knitted garment of claim 1, wherein the garment is a pair of men's tights, wherein the first compression zone is located in a genital area, and wherein the second compression zone is located in an area surrounding the genital area.

20. The engineered knitted garment of claim 1, wherein the garment is a bra, wherein the first compression zone is located in an areola area, and wherein the second compression zone is located in an area surrounding the areola area.

21. The engineered knitted garment of claim 1, wherein the garment is a women's bathing suit, wherein the second compression zone is located in an central abdominal area, and wherein the first compression zone is located in an area surrounding the central abdominal area.

22. The engineered knitted garment of claim 1, wherein the garment is a pair of women's underwear, wherein the second compression zone is located in a pelvic area, and wherein the first compression zone is located in a waistband area.
